(19) United States
(12) Patent Application Publication
(10) Pub. No.: US 2023/0380992 A1
(43) Pub. Date: Nov. 30, 2023

(54) Prosthetic Limb Systems and Devices

(71) Applicant: Cristian Rogers, Irvine, CA (US)

(72) Inventor: Cristian Rogers, Irvine, CA (US)

(73) Assignee: Paeon, Inc., Irvine, CA (US)

(21) Appl. No.: 17/751,674

(22) Filed: May 24, 2022

(51) Int. Cl. A61F 2/58

(52) U.S. Cl. CPC A61F 2/583; A61F 2002/543

(57) ABSTRACT

An adjustable prosthetic arm powered by mechatronic systems capable of osseointegration based and non-osseointegration based attachment via adjustable socket.

### FIELD OF THE INVENTION

The field of the invention is adjustable prosthetic limbs.

### BACKGROUND

The background description includes information that may be useful in understanding the present invention. It is not an admission that any of the information provided in this application is prior art or relevant to the presently claimed invention, or that any publication specifically or implicitly referenced is prior art.

Traditionally a prosthetic limb is a device that consists of two components a hand (or foot) and a socket for the residual limb which requires a prosthetist (a clinician that takes measurement of the amputees residual limb and later constructs an indeterminate amount of protype molds and/or prints of the residual limb) and specific measurements to construct the device. The final device is almost always constructed by two or more parties if the prosthetic hand or foot has mechanical properties.

These and all other extrinsic materials discussed in this application are incorporated by reference in their entirety. Where a definition or use of a term in an incorporated reference is inconsistent or contrary to the definition of that term provided in this application, the definition of that term provided in this application applies and the definition of that term in the reference does not apply.

It has yet to be appreciated that amputees need a prosthesis that can be used immediately after limb amputation for trauma amputees or in the development stage after birth for congenital amputees.

Thus, there is still a need in the art for an adjustable prosthetic arm powered by mechatronic systems.

### BRIEF DESCRIPTION OF THE DRAWING

FIG. 1A is a posterior view of an adjustable prosthetic limb with a hand in an open configuration and with a socket in a closed configuration.

FIG. 1B is an anterior view thereof with the hand in an open configuration and with the socket in a closed configuration.

FIG. 2A is a lateral view thereof with the socket in a closed configuration.

FIG. 2B is a medial view of thereof with the socket in a closed configuration.

FIG. 3A is a lateral view of a prosthetic limb inner liner.

FIG. 3B is a medial view of the inner liner.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used in the description in this application and throughout the claims that follow, the meaning of “a,” “an,” and “the” includes plural reference unless the context clearly dictates otherwise. Also, as used in the description in this application, the meaning of “in” includes “in” and “on” unless the context clearly dictates otherwise.

Also, as used in this application, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

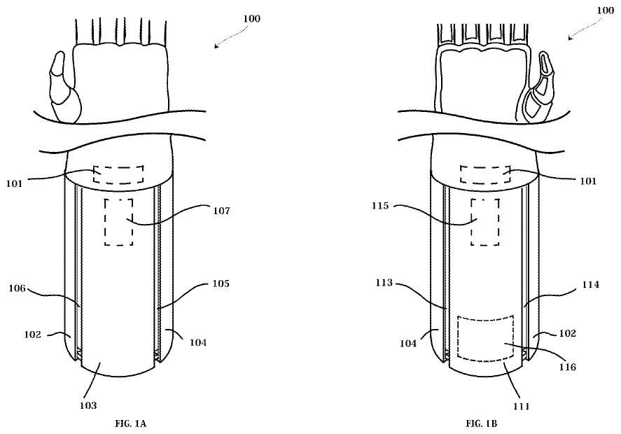

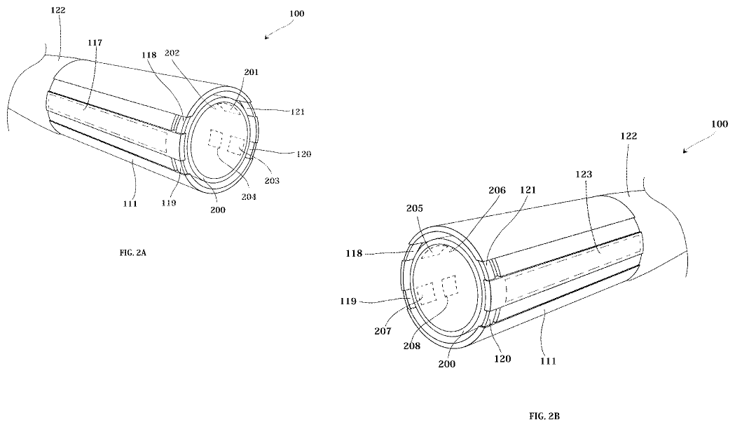

In FIG. 1A, a prosthetic device 100 of the inventive subject matter is shown. Prosthetic device 100 features a forearm and prosthetic hand (shown in FIGS. 1A and 1B), and several of its moving components (e.g., fingers) can be actuated by one or more electric motors (e.g., servo motors). FIG. 2A, which shows another view of prosthetic device 100 without showing the hand, shows muscle sensor regions 207 and 210, (which are comprised of, e.g., electromyography sensors, etc.) as well as a user input region illustrated by component 111 and 115 in FIG. 1B. Components 111 and 115 represent user input regions of prosthetic device 100, and the components can include, e.g., physical buttons or a touch-sensitive display. Adjustable prosthetic device 100 is shown in FIGS. 1A-2B. Component 101 is an internal electric motor (shown in broken lines to indicate that it exists on an interior portion of the device as shown in FIG. 1B) that is configured to actuate the panel components 102 and 104, which pivot in relation to the wrist component 122 (such that the distal end of the panels act as a hinge allowing the proximal end of the panels to be actuated) and house cable systems 117 and 123 that are actuated by the electric motor 101 that in turn actuate the panels 102 and 104. When panels 102 and 104 are actuated they actuate panels 103 and 111 by guiding them with the rails 118 through 120 by changing the length of cables (e.g., shortening or lengthening) within the region illustrated by component 117 in FIG. 2A and 123 in FIG. 2B.

In FIG. 1B, the illustrated user input region displayed by component 111 and 115 is a multifunction component which is used to but not limited to powering on or off the device, selecting a grip preset, and checking battery life. A user can receive feedback from prosthetic device 100 via, e.g., an indicator light region 107 and/or via a mobile application. Prosthetic device 100 can receive power by a wired connection or by a wireless connection in via power input region 116 shown in, e.g., FIG. 1B.

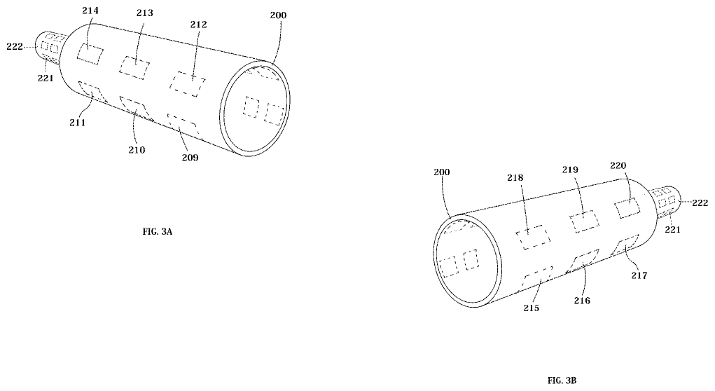

As shown in FIG. 2A, the prosthetic socket is comprised of components that allow it to increase and decrease a proximal diameter of the elastic inner liner or elastic grip. Elastic liner 200 can be made from a rubber, fabric and/or any other synthetic material. Elastic liners of the inventive subject matter can also be used to, e.g., increase and decrease a diameter between the components 102 through 106, and 111 in FIGS. 1A and 1B. A diameter of a distal portion of those components are not altered in the same fashion because they are hinged to component 122 as shown in FIG. 2A. The outer panels mentioned above are actuated by the electric motor and cable systems 101 117, and 123 in FIGS. 1A, 2A and 2B and guided by inner panel components 105, 106, 113, 114, in FIGS. 1A and 1B as well as by the rail components 118 through 121 in FIG. 2A. The outer panels, inner panels, electric motor, and rails mentioned above construct the adjustable socket of the adjustable prosthesis. Which allows the amputee to alter the prosthesis for a desired fit on their residual limb. This can be accomplished via component 200, 115 and/or a mobile application. The prosthetic device 100 will be held on to the residual limb with component 200, which has a size relative to the diameter of components 102 through 106, 111, 113 and 114 (e.g., such that if electric motor 101 actuates components 102 through 106, 111, 113, and 114 by shortening the cables with cable systems 117 and 123 then the diameter between the components would decrease and the diameter between those components would increase in the case of electric motor denoted component 101 lengthening those cables), the amputee controls component 100, the prosthetic hand, using muscle sensor regions 201 through 208 and 212 through 215, which are used to collect the electrical signals produced by the amputee's residual limb, converting the analog signals into digital signals that are then interpreted as movement in the prosthetic device (e.g., the hand) the data captured in these regions may be captured using but not limited to electromyography sensors that capture the electrical signals produce by the amputee's residual limb muscles. The elastic inner liner or elastic grip is anchored to the adjustable prosthetic socket components 105, 106, 113, 114, in FIG. 1A in regions illustrated by components 209 through 220 in FIG. 3A and FIG. 3B. The elastic inner liner is anchored to component 122 in FIGS. 2A and 2B using component 221 and 222 in FIGS. 3A and 3B.

Thus, specific compositions and methods of adjustable prosthetic limb have been disclosed. It should be apparent, however, to those skilled in the art that many more modifications besides those already described are possible without departing from the inventive concepts in this application. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A prosthetic device, comprising:
a sleeve; and
a forearm and a prosthetic hand.
